(19) United States
(12) Patent Application Publication
(10) Pub. No.: US 2009/0075948 A1
(43) Pub. Date: Mar. 19, 2009

(54) COMPOSITIONS AND METHODS FOR REGULATING CELLULAR PROTECTION

(75) Inventor: CARL S. PARKER, Altadena, CA (US)

Correspondence Address: KAREN S. CANADY;CANADY & LORTZ LLP, COMMERCE PLAZA, 11340 WEST OLYMPIC BLVD., SUITE 275, LOS ANGELES, CA, 90064, US

(73) Assignee: California Institute of Technology, Pasadena, CA (US)

(21) Appl. No.: 12/210,206

(22) Filed: Sep. 14, 2008

Related U.S. Application Data
(60) Provisional application No. 60/993,845, filed on Sep. 14, 2007.

(51) Int. Cl. A61K 31/60; C12N 5/00; G01N 33/566; A61P 25/28

(52) U.S. Cl. 514/159; 435/375; 435/7.2

(57) ABSTRACT

The invention provides a method of inducing a protective response in cells by activating a heat shock response, via heat shock transcription factor (HSF), without stressing the cells. The invention is based on the surprising discovery that HSF can be activated in non-stressed cells by disrupting interaction with the repressive cognate protein, Hsc70. As described herein, non-steroidal anti-inflammatory drugs (NSAID), including salicylic acid and ibuprofen, can stimulate HSF DNA binding, Hsp70 transcription and Hsp70 protein synthesis in non-stressed cells. By activating HSF, the degenerative effects of certain neurodegenerative proteins such as polyglutamine repeat proteins (Huntington's disease) and the α-synuclein protein (Parkinson's disease) can be offset. The invention thus provides a method of ameliorating symptoms of neurodegenerative disease.

This application claims benefit of U.S. provisional patent application Ser. No. 60/993,845, filed Sep. 14, 2007, the entire contents of which are incorporated herein by reference.

### TECHNICAL FIELD

The present invention relates generally to the prevention and therapy of neurodegenerative diseases. More specifically, the invention relates to the protection of cells against heat and denatured or damaged proteins. Such proteins accumulate in the neurons of a wide variety of neurodegenerative diseases. The self-defense mechanism used by cells is employed to counteract the damage inflicted on neurons by the disease proteins. Cells treated with the agents of the invention respond by activating a transcription factor known as the heat shock transcription factor (HSF), which in turn activates the synthesis of protective heat shock proteins including hsp70.

### BACKGROUND OF THE INVENTION

All organisms from bacteria to man can sense temperature. At the center of temperature sensing is a single transcription factor known as the heat shock transcription factor (HSF). How this factor senses temperature and is regulated is not understood. In the past, efforts were made to demonstrate that the HSF itself could respond to temperature in a direct way in vitro (Rabindran et al. 1991; Sarge et al. 1993; Rabindran et al. 1994; Zuo et al. 1994; see list of cited references at end of Example 1) by acting as the cell's ‘thermometer’. Several investigators addressed the question as to whether HSF could respond to temperature as an intrinsic function of the factor without cofactors. Recombinant mammalian HSF when over expressed and purified from E. coli was shown to be somewhat heat-inducible if diluted to the appropriate level (Rabindran et al. 1993; Zuo et al. 1994; Goodson and Sarge 1995; Farkas et al. 1998). The same was also shown for Drosophila HSF, yet in both cases the time required to bind DNA was very lengthy (30 minutes) and the fractional amount of HSF actually able to bind DNA small (Zhong et al. 1998). In the cell, activation of DNA binding occurs rapidly and robusty; 1-2 minutes, and the HSF is fully activated to bind to the heat shock protein (Hsp) promoters (Lindquist and Craig 1988). Apparently, purified recombinant HSF is lacking essential co-factors necessary to carry out its mission of rapidly responding to temperature shock and activating transcription of the Hsp genes.

I have purified a Drosophila and human HSF complex (HSF-C) using tandem affinity purification methods (TAP). Present in both of these complexes is a well-known Hsp cognate protein known as Hsc4p in Drosophila and Hsc70(8) in human cells. I knocked down the expression specifically of the Hsc4p cognate in Drosophila and found that the HSF was fully active in DNA binding and transcription. Thus Hsc4p in Drosophila functions to repress the activity in normally growing cells. In human cells, another laboratory demonstrated that knocking down Hsc70 activated DNA binding of HSF in normally growing cells, but they concluded that this was not significant because the level of DNA binding was very low, as was the level of hsp70 synthesis. “The observed low levels may represent an insignificant difference between samples, but, more likely, the result suggest that the cells, using a currently unknown mechanism, were attempting to compensate for the loss of Hsc70.” (Ahn et al. 2005) In Drosophila when the HSF is activated it binds to DNA, activates transcription and is degraded. It is likely that this true for the human HSF, however, Ahn, et al. did not examine the levels of HSF in the knock down experiment. Active HSF levels drop significantly when Hsc4p is knocked down in non-stressed cells and this would explain the low level of DNA binding observed in human cells.

There remains a need to identify the domain of HSF that interacts with Hsc4p and disrupt this interaction as a means to activate HSF. The potential value of activating the HSF in non-stressed cells without stress is significant. Hsp70 can offset the degenerative effects of certain neurodegenerative proteins such as polyglutamine repeat proteins in Huntington's disease and the α-synuclein protein in Parkinson's disease. Thus inducing the synthesis of Hsp70 in the absence of cellular stress by activating HSF could have great value in treating these and other diseases.

### SUMMARY OF THE INVENTION

The invention provides a method of increasing Hsp70 synthesis in a cell in the absence of stress. In one embodiment, the method comprises contacting the cell with an effective amount of an agent that disrupts the interaction of Hsc70 with heat shock transcription factor (HSF). The invention further provides a method of activating a heat shock response in a cell in the absence of stress. The method comprises contacting the cell with an effective amount of an agent that disrupts the interaction of Hsc70 with HSF. In one embodiment, the agent comprises a non-steroidal anti-inflammatory drug (NSAID), such as salicylic acid or ibuprofen. In a typical embodiment of the methods of the invention, the agent disrupts binding between Hsc70 and the oligomerization domain of HSF. In one embodiment, the agent comprises an RNAi directed at Hsc70. The heat shock response can comprise, for example, increase binding of heat shock transcription factor (HSF) to DNA, increased transcription of Hsp70 and/or increased synthesis of Hsp70. The cell can be in vivo or in vitro. In a typical embodiment, the contacting occurs while the cell is maintained at a temperature of 28 to 39° C., most typically, about 37° C. In one embodiment, the effective amount of agent, such as an NSAID, is between 0.01 and about 10 mM, typically, about 0.5 to about 3 mM. The contacting can occur, for example, for about 1 to about 90 minutes, typically for about 60 minutes.

The invention further provides a method of protecting cells from neurodegenerative proteins in the brain of a mammalian subject. The method comprises administering to the subject an effective amount of a non-steroidal anti-inflammatory drug (NSAID), wherein the amount of NSAID is sufficient to increase binding of heat shock transcription factor (HSF) to DNA, increase transcription of Hsp70, or to increase synthesis of Hsp70 in brain cells of the subject. In a typical embodiment, the administering is oral.

The invention additionally provides a method of identifying an agent that protects neural cells from neurodegenerative proteins. The method comprises contacting cultured cells with a candidate agent; and assaying the cultured cells for levels of Hsp70 protein. An increase in levels of Hsp70 protein is indicative of a neuroprotective agent. The cells are typically mammalian cells or Drosophila cells. In one embodiment, the cultured cells contain a reporter construct that responds to activation of Hsp70 synthesis by producing a detectable signal and the assaying comprises measuring levels of the detectable signal. The reporter construct can be, for example, a plasmid. One example of a detectable signal is fluorescence.

### BRIEF DESCRIPTION OF THE FIGURES

FIG. 1. Western analysis of Drosophila Heat shock Factor TAP. Lane 1: S10 preparation; lane 2: supernatant of IgG Agarose resin; lane 3: nuclear extract; lane 4: elution by TEV cleavage from the IgG resin; lane 5: supernatant of the CM Sepharose resin; lanes 7-14: fractions eluted from the CM Sepharose resin.

FIG. 2A. Ag-stained SDS-PAGE of Drosophila HSF-TAP. Lane 1: S10; lane 2: supernatant of IgG Agarose resin; lane 3: eluate from IgG resin; lane 4: supernatant of CM Sepharose resin; lanes 5-9: elution from CM Sepharose resin.

FIG. 2B. Coomassie stain of Dm HSF-C purified by TAP.

FIG. 2C. TAP of HSF-1 from human 293 cells transfected with pcDNA6-HSF-1. Coomassie stained gel. Control lane Human 293 cells not transfected with HHSF-1 DNA. HHSF-1-C TAP preparation from transfected cells. Mr=molecular weight marker.

FIG. 3A. Specificity of RNAi KD using 3′-UTR-specific directed RNAi. Western blot with antibodies reactive against Hsc70 and Hsp70. Lanes 1 and 3 non-shocked cells. Lanes 2 and 4 heat shocked cells. Lanes 1 and 2 are extracts prepared from cells treated with Hsc4p 3′-UTR RNAi for four days. Lanes 3 and 4 are extracts prepared from cells treated with Hsc3p 3′-UTR RNAi for four days.

FIG. 3B. Effects of 3′-UTR RNAi targeting Hsc3p on the expression of Hsc3p and Hsc4p using an antibody reactive against Hsc3p. Lanes 1 and 2 non-shocked cells. Lanes 2 and 4 heat shocked cells. Lanes 1 and 2 cells were treated with RNAi against Hsc4p 3′-UTR. Lanes 3 and 4 cells were treated with RNAi against Hsc3p 3′-UTR.

FIG. 4. HSF-DNA binding activity of S2 cells with RNAi KD and gene KI: comparison of normal cells with heat shocked cells. All odd numbered lanes are non-shocked cells and all even numbered lanes are heat-shocked cells. Lanes 1 and 2: control cells. Lanes 3 and 4: Hsc3p knock down with RNAi. Lanes 5 and 6: Hsc4p knock down with RNAi. Lanes 7 and 8: knock-in Hsc4p (wt). Lanes 9 and 10 knock-in Hsc4p (K71S). Lanes 11 and 12: knock-in Hsc4p (D206S). Lanes 13 and 14: knock-down of Chip with RNAi. HSF/HSE: specific complexes of HSF and HSE. NS: non-specific complex can serve as a control for comparing overall protein levels.

FIG. 5. Primer-extension analysis of hsp70 transcription in normal and heat-shocked cells treated with RNAi (KD). All odd numbered lanes, non-shocked cells; all even numbered lanes, heat-shocked cells. Lanes 1 and 2, control cells. Lanes 3 and 4, KD with RNAi against Hsc4p. Lanes 5 and 6, KD with RNAi against Hsc3p.

FIG. 6. Effects on Hsp70 transcription with Hsc4p KI. A comparison of wild-type Hsc4p with dominant-negative mutants K71S and D206S. All odd numbered lanes are non-shocked. All even numbered lanes are heat shocked. Lanes 1 and 2 are Ksc4p (wt) KI. Lanes 3 and 4 are Hsc4p (K71S) KI. Lanes 5 and 6 are Hsp4p (D206S) KI. The control cells for this experiment are in the previous FIG. 6.

FIG. 7. Western analysis of the effects of Hsc3p, Hsc4p and CHIP KD with RNAi and the effects of Hsc4p wt, K71S and D206S KI on normal and heat shocked S2 cells. All odd-numbered lanes are non-shocked cells. All even numbered lanes are heat shocked cells. Lanes 1 and 2 are control cells. Lanes 3 and 4, Hsc3p KD. Lanes 5 and 6, Hsc4p KI. Lanes 7 and 8, CHIP KD. Lanes 9 and 10, Hsc4p (wt) KI. Lanes 11 and 12, Hsc4p (K71S) KI. Lanes 13 and 14, Hsc4p (D206S) KI.

FIG. 8. Co-immunoprecipitation of Hsc70 with HSF in non-shocked and heat shocked cells. Lanes 1 and 3 are the supernatants of the ip with anti-HSF antibody. Lanes 2 and 4 are the ip samples with anti-HSF antibodies. Lanes 1 and 2 are samples from non-shocked cells. Lanes 3 and 4 are samples from heat-shocked cells.

FIG. 9. DNA binding activity of purified HSF-C, activation of DNA binding by temperature and Hsc4p. Lanes 1-3 non shocked in vitro. Lanes 46 heat shocked in vitro for 3 minutes. Lanes 1 and 4 HSF-C only. Lanes 2 and 5 Hsc4p purified from S2 cells. Lanes 3 and 6 contain a combination of HSF-C and Hsc4p.

FIG. 10. Effect of heat shock on the composition of HSF-C. Cytosolic (C) and nuclear (N) fractions from non-shocked and heat shocked cells analyzed by western blotting, probed with antibodies against HSF, Hsc3p and Hsc4p.

FIGS. 11A-11B. Binding of Hsc4p/3p to internal deletions of the oligomerization domain of HSF. FIG. 11A shows the S35 HSF molecules used in the binding reactions. FIG. 11B shows a defined view of the interactions between the two cognates, Hsc4p and Hsc3p, and the oligomerization domain of the HSF. The SDS gel shows an important interaction occurs between Hsc4p and D7 (residues 181-210), while Hsc3p has a broader interaction with D7 and D8 (residues 211-240) although the D8 interaction is less strong than D7.

FIGS. 12A-12B. HSF binding domains on Hsc4p and Hsc3p. Input to the binding reactions is shown in FIG. 12A. The results of protein binding are shown in FIG. 12B. Lanes 1-4 contain Hsc4p and lanes 5-8 contain Hsc3p. Lanes 1 and 5 contain full length proteins, lanes 2 and 6 contain carboxyl terminal truncations at residues 506 and 501 respectively. Lanes 3 and 7 contain carboxyl terminal truncations at residues 406 and 410, respectively. Lanes 4 and 8 contain carboxyl terminal truncations at residues 306 and 311, respectively. Clearly HSF binds to the amino terminal portion of both cognates, specifically the first 300 amino acids that represents the ATPase domains of the proteins.

FIG. 13. Protein/protein interaction analysis of HSF binding domain for Hsc4p and Hsc3p. Upper panel is HSF COOH truncation input; middle panel is HSF binding to Hsc4p; lower panel is HSF binding to Hsc3p. Lanes 1 (all 3 panels) contain full length HSF; lanes 2 contain HSF truncated at residue 645; lanes 3 contain HSF truncated at residue 545; lanes 4 contain HSF truncated at residue 445; lanes 5 contain HSF truncated at residue 345; lanes 6 contain HSF truncated at residue 245; lanes 7 contain HSF truncated at residue 145.

FIG. 14. Induction of HSF DNA binding and hsp70 protein synthesis by ibuprofen and salicylic acid. The concentrations of ibuprofen and salicylic acid are shown as mM. Both DNA binding and Hsp70 protein synthesis follow the same optimal concentrations. The cells were treated for 60 minutes with the indicated drug and whole cell extracts prepared for analysis.

FIG. 15. Time course for Hsp70 transcription induction by 3 mM salicylic acid. Cells were treated with 3 mM salicylic acid and RNA was isolated at the indicated times. Hsp70 transcription was induced rapidly, comparable to that of heat shock; within one minute Hsp70 transcripts are detectable.

FIG. 16. Purification of HSF complex from human and fly cells.

FIGS. 17A-17C. Effects of knocking down Hsp3p (Hsc72/Bip) and Hsc4p (Hsc70) on the heat shock response in Drosophila in vivo. FIG. 17A: DNA binding activity of HSF measured by electrophoretic mobility-shift assay (EMSA). FIG. 17B: Hsp70 transcription monitored by primer extension analysis. FIG. 17C: HSF levels are dramatically reduced when Hsc4p is knocked down. This series of western blots demonstrates the specificity of the individual knock downs and presents a control of protein levels with Kap-a3 nuclear transporter molecule.

### DETAILED DESCRIPTION OF THE INVENTION

The invention provides a method of inducing a protective response in cells by activating a heat shock response, via heat shock transcription factor (HSF), without stressing the cells. The invention is based on the surprising discovery that HSF can be activated in non-stressed cells by disrupting interaction with the repressive cognate protein, Hsc70. As described herein, non-steroidal anti-inflammatory drugs (NSAID), including salicylic acid and ibuprofen, can stimulate HSF DNA binding, Hsp70 transcription and Hsp70 protein synthesis in non-stressed cells. By activating HSF, the degenerative effects of certain neurodegenerative proteins such as polyglutamine repeat proteins (Huntington's disease) and the α-synuclein protein (Parkinson's disease) can be offset. The invention thus provides a method of ameliorating symptoms of neurodegenerative disease.

### DEFINITIONS

All scientific and technical terms used in this application have meanings commonly used in the art unless otherwise specified. As used in this application, the following words or phrases have the meanings specified.

As used herein, “heat shock” refers to the effect of subjecting a cell to a higher temperature than that of the ideal body temperature of the organism from which the cell was derived. Heat shock is a form of stress, as is cold or oxygen deprivation. The “absence of stress”, in this context, means without exposing a cell to a temperature above or below its normal temperature (e.g., ideal body temperature) or depriving it of oxygen.

As used herein, “heat shock transcription factor” or “HSF” refers to a transcription factor found in all eukaryotic cells that responds to temperature stress to activate the transcription of protective proteins collectively know as the heat shock proteins. The HSF is generally found as a single protein but some organisms possess several other forms of HSF that are specialized and not temperature responsive, all organisms have at least one HSF that is temperature responsive. In the examples presented below, reference is to the temperature responsive HSF known as HSF-1 in human cells.

As used herein, “RNAi” or “RNA interference” refers to a mechanism that inhibits gene expression at the stage of translation or by hindering the transcription of specific genes. The specific RNAi methods described in the examples below block the translation of the targeted message by destroying the 3′ untranslated leader, most likely preventing polyadenylation and proper stabilization of the mRNA. Specific targeting of the 3′-untranslated region provides specificity in the knockdown because Hsc3p and the Hsc4p message bear little similarity in those regions of the mRNA. As used herein, “polypeptide” includes proteins, fragments of proteins, and peptides, whether isolated from natural sources, produced by recombinant techniques or chemically synthesized. Polypeptides of the invention typically comprise at least about 6 amino acids.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, to “prevent” or “treat” a condition means to decrease or inhibit symptoms indicative of the condition or to delay the onset or reduce the severity of the condition.

As used herein, “a” or “an” means at least one, unless clearly indicated otherwise.

### Methods of the Invention

The invention provides a method of activating a heat shock response in a cell in the absence of stress. The method comprises contacting the cell with an effective amount of an agent that disrupts the binding of Hsc70 to HSF. The binding of Hsc70 to HSF can be disrupted by targeting the oligomerization domain of HSF, between amino acid residues 146 and 245, more specifically, residues 181 to 240.

In a typical embodiment, the agent is a non-steroidal anti-inflammatory drug (NSAID). Representative NSAIDs include, but are not limited to, salicylic acid, ibuprofen, aspirin, indomethacin, nabumetone, diclofenac, piroxicam, phenyl butazone, sulindac and meclofenamic acid. A typical agent is one that can induce HSF DNA binding (Soncin and Calderwood BBRC 1996, 229: 479-484).

The heat shock response to be activated can comprise increased binding of heat shock transcription factor (HSF) to DNA, increased transcription of Hsp70, and/or increased synthesis of Hsp70. Activating the heat shock response protects cells, including mammalian brain cells, from neurodegenerative proteins. This activation can be used to treat neurodegenerative disease and ameliorate symptoms of such diseases, as well as to generally improve cellular protection. Representative neurodegenerative diseases whose symptoms can be ameliorated by the invention include, but are not limited to, Huntington's disease, Parkinson's disease, Alzheimer's disease, frontotemporal dementia with parkinsonism and trinucleotide expansion diseases of type I (polyQ) diseases. Examples of neurodegenerative proteins include, but are not limited to, polyglutamine repeat proteins and α-synuclein protein.

The method of the invention can be performed in vivo, such as by systemic treatment of a mammalian subject, or in vitro, by direct contact with cells to be treated. In some embodiments, the contacting occurs while the cell is maintained at a temperature of 28 to 39° C., typically at about 37° C. In one embodiment, the effective amount of NSAID is between 0 and about 10 mM. In some embodiments, the effective amount of NSAID is about 1 to about 3 mM. The contacting can occur for about 1 to about 90 minutes, or longer, but typically occurs for about 60 minutes. In some embodiments, the method is designed to provide protection on a continuous basis, such as by maintaining a minimal level of hsp70 induction.

The invention additionally provides a method of protecting cells from neurodegenerative proteins in the brain of a mammalian subject. Also provided are methods of treating neurodegenerative disease, ameliorating symptoms of such diseases, and improving cellular protection against stress, without subject the cells or the subject to stress. The methods comprise administering to the subject an effective amount of a non-steroidal anti-inflammatory drug (NSAID) or other agent that activates the heat shock response under non-stress conditions, such as by disrupting interaction between Hsc70 and HSF, by increased binding of heat shock transcription factor (HSF) to DNA, increased transcription of Hsp70, and/or increased synthesis of Hsp70.

The subject can be a mammal, preferably a human. Veterinary subjects, such as canine, equine, feline, ovine, porcine, bovine or other subjects are within the scope of the invention. The NSAID can be administered by well-known means, including injection (e.g., intracutaneous, intratumoral, intramuscular, intravenous or subcutaneous), intranasally (e.g., by aspiration) or orally.

### Polynucleotides of the Invention

The invention provides polynucleotides, such as can be used to disrupt repressor (Hsc70) binding to heat shock transcription factor (HSF), or a portion or other variant thereof. Preferred polynucleotides comprise at least 15 consecutive nucleotides, preferably at least 20 consecutive nucleotides and more preferably at least 25 consecutive nucleotides, that target a domain of interest on HSF, such as the oligomerization domain (between amino acid residues 146 and 245, more specifically, residues 181 to 240). Polynucleotides complementary to any such sequences are also encompassed by the present invention. Polynucleotides may be single-stranded (coding or antisense) or doublestranded, and may be DNA (genomic, cDNA or synthetic) or RNA molecules. RNA molecules include siRNA molecules, HnRNA molecules, and mRNA molecules. Additional coding or non-coding sequences may, but need not, be present within a polynucleotide of the present invention, and a polynucleotide may, but need not, be linked to other molecules and/or support materials.

Polynucleotides may comprise a native sequence or may comprise a variant of such a sequence. Polynucleotide variants contain one or more substitutions, additions, deletions and/or insertions such that the activity of the polynucleotide or its encoded polypeptide is not diminished, relative to a native protein. Variants preferably exhibit at least about 70% identity, more preferably at least about 80% identity and most preferably at least about 90% identity to a polynucleotide sequence that encodes a native protein or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor. 11:105; Santou, N., Nes, M. (1987) Mol. Biol. Evol. 4:406-425; Sneath, P. H. A. and Sokal, R. R. (1973) Numerical Taxonomy the Principles and Practice of Numerical Taxonomy, Freeman Press, San Francisco, Calif.; Wilbur, W. J. and Lipman, D. J. (1983) Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native protein (or a complementary sequence). Suitable moderately stringent conditions include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Polynucleotides may be prepared using any of a variety of techniques known in the art. DNA may be obtained from a cDNA library. The polynucleotide may also be obtained from a genomic library or by oligonucleotide synthesis. Libraries can be screened with probes (such as antibodies or oligonucleotides of at least about 20-80 bases) designed to identify the gene of interest or the protein encoded by it. Illustrative libraries include human liver cDNA library (human liver 5′ stretch plus cDNA, Clontech Laboratories, Inc.) and mouse kidney cDNA library (mouse kidney 5-stretch cDNA, Clontech laboratories, Inc.). Screening the cDNA or genomic library with the selected probe may be conducted using standard procedures, such as those described in Sambrook et al., Molecular Cloning: A Laboratory Manual (New York: Cold Spring Harbor Laboratory Press, 1989). An alternative means to isolate the gene encoding is to use PCR methodology (Sambrook et al., supra; Dieffenbach et al., PCR Primer A Laboratory Manual (Cold Spring Harbor Laboratory Press, 1995)).

The oligonucleotide sequences selected as probes should be sufficiently long and sufficiently unambiguous that false positives are minimized. The oligonucleotide is preferably labeled such that it can be detected upon hybridization to DNA in the library being screened. Methods of labeling are well known in the art, and include the use of radiolabels, such as ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined through sequence alignment using computer software programs, which employ various algorithms to measure homology.

Nucleic acid molecules having protein coding sequence may be obtained by screening selected cDNA or genomic libraries, and, if necessary, using conventional primer extension procedures as described in Sambrook et al., supra, to detect precursors and processing intermediates of mRNA that may not have been reverse-transcribed into cDNA.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (see Adelman et al., DNA 2:183, 1983). Alternatively, RNA molecules may be generated by in vitro or in vivo transcription of DNA sequences encoding a protein, or portion thereof, provided that the DNA is incorporated into a vector with a suitable RNA polymerase promoter (such as T7 or SP6). Certain portions may be used to prepare an encoded polypeptide, as described herein.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences can be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and to permit expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

### HSF Polypeptides

Within the context of the present invention, HSF polypeptides comprise at least the oligomerization domain (between amino acid residues 146 and 245, more specifically, residues 181 to 240) and/or a variant thereof. Polypeptides as described herein may be of any length. Additional sequences derived from the native protein and/or heterologous sequences may be present. In some embodiments, the HSF polypeptide comprises amino acid residues 181 to 210 or 211 to 240.

Polypeptide variants preferably exhibit at least about 70%, more preferably at least about 90% and most preferably at least about 95% identity (determined as described above) to the identified polypeptides.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

Polypeptides may comprise a signal (or leader) sequence at the N-terminal end of the protein that co-translationally or post-translationally directs transfer of the protein. The polypeptide may also be conjugated to a linker or other sequence for ease of synthesis, purification or identification of the polypeptide (e.g., poly-FEs), or to enhance binding of the polypeptide to a solid support. For example, a polypeptide may be conjugated to an immunoglobulin Fc region.

Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast, insect cells or a mammalian cell line such as COS or CHO. Supernatants from suitable host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Polypeptides can be synthesized on a Perkin Elmer/Applied Biosystems Division 430A peptide synthesizer using FMOC chemistry with HPTU (O-BenzotriazoleN,N,N′,N′-tetramethyluronium hexafluorophosphate) activation. A Gly-Cys-Gly sequence may be attached to the amino terminus of the peptide to provide a method of conjugation, binding to an immobilized surface, or labeling of the peptide. Cleavage of the peptides from the solid support may be carried out using the following cleavage mixture: trifluoroacetic acid:ethanedithiol:thioanisole:water:phenol (40:1:2:2:3). After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0%-60% acetonitrile (containing 0.1% TFA) in water may be used to elute the peptides. Following lyophilization of the pure fractions, the peptides may be characterized using electrospray or other types of mass spectrometry and by amino acid analysis.

### Fusion Proteins

In some embodiments, the polypeptide is a fusion protein that comprises multiple polypeptides as described herein, or that comprises at least one polypeptide as described herein and an unrelated sequence. A fusion partner may, for example, serve as an expression enhancer, assisting in expressing the protein at higher yields than the native recombinant protein. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues, that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located 5′ to the DNA sequence encoding the first polypeptides. Similarly, stop codons required to end translation and transcription termination signals are present 3′ to the DNA sequence encoding the second polypeptide.

In general, polypeptides (including fusion proteins) and polynucleotides as described herein are isolated. An “isolated” polypeptide or polynucleotide is one that is removed from its original environment. For example, a naturally occurring protein is isolated if it is separated from some or all of the coexisting materials in the natural system.

Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### Pharmaceutical Compositions

The invention provides small molecules that are incorporated into pharmaceutical compositions. Pharmaceutical compositions comprise one or more such compounds and, optionally, a physiologically acceptable carrier. Pharmaceutical compositions within the scope of the present invention may also contain other compounds that may be biologically active or inactive.

The small molecule or other compound is an agent that disrupts the binding of Hsc70 to HSF. In a typical embodiment, the agent is a non-steroidal ant-inflammatory drug (NSAID). Representative NSAIDs include, but are not limited to, salicylic acid, ibuprofen, aspirin, indomethacin, nabumetone, diclofenac, piroxicam, phenyl butazone, sulindac and meclofenamic acid. A typical agent is one that can induce HSF DNA binding (Soncin and Calderwood BBRC 1996, 229: 479-484).

A pharmaceutical composition can contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guemn) that expresses an immunogenic portion of the polypeptide on its cell surface or secretes such an epitope.

In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques 6:615-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. A typical embodiment comprises oral administration and is formulated to facilitate crossing the blood-brain barrier. For oral administration, any of the above carriers or a solid carrier, such as mannitol, lactose, starch, magnesium stearate, sodium saccharine, talcum, cellulose, glucose, sucrose, and magnesium carbonate, may be employed. For parenteral administration, such as subcutaneous injection, the carrier preferably comprises water, saline, alcohol, a fat, a wax or a buffer. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

### Therapeutic and Prophylactic Methods

The small molecules and pharmaceutical compositions of the invention can be administered to a subject, thereby providing methods for inhibiting neurodegenerative proteins, and for the treatment or prevention of neurodegenerative disease.

Treatment includes prophylaxis and therapy. Prophylaxis or therapy can be accomplished by a single direct injection at a single time point or multiple time points to a single or multiple sites. Administration can also be nearly simultaneous to multiple sites.

Patients or subjects include mammals, such as human, bovine, equine, canine, feline, porcine, and ovine animals. The subject is preferably a human, and may or may not be afflicted with disease.

In some embodiments, the condition to be treated or prevented is a neurodegenerative disease. The treatment can be used to ameliorate symptoms of such diseases, as well as to generally improve cellular protection. Representative neurodegenerative diseases whose symptoms can be ameliorated by the invention include, but are not limited to, Huntington's disease, Parkinson's disease, Alzheimer's disease, frontotemporal dementia with parkinsonism and trinucleotide expansion diseases of type I (polyQ) diseases. Examples of neurodegenerative proteins include, but are not limited to, polyglutamine repeat proteins and α-synuclein protein.

### Administration and Dosage

The compositions are administered in any suitable manner, often with pharmaceutically acceptable carriers. Suitable methods of administering cells in the context of the present invention to a subject are available, and, although more than one route can be used to administer a particular cell composition, a particular route can often provide a more immediate and more effective reaction than another route.

The dose administered to a patient, in the context of the present invention, should be sufficient to effect a beneficial therapeutic response in the patient over time, or to inhibit disease. Thus, the composition is administered to a subject in an amount sufficient to elicit an effective response to activate hsp70 synthesis and/or to alleviate, reduce, cure or at least partially arrest symptoms and/or complications from the disease. An amount adequate to accomplish this is defined as a “therapeutically effective dose.”

Routes and frequency of administration of the therapeutic compositions disclosed herein, as well as dosage, will vary from individual to individual, and may be readily established using standard techniques. In general, the pharmaceutical compositions may be administered, by injection (e.g., intracutaneous, intratumoral, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. In one embodiment, 2 intradermal injections of the composition are administered 10 days apart.

A suitable dose is an amount of a compound that, when administered as described above, is capable of promoting a detectable response, and is at least 10-50% above the basal (i.e., untreated) level. Such response can be monitored, for example, by measuring indicators of an improved clinical outcome (e.g., more frequent remissions, complete or partial or longer disease-free survival) in treated patients as compared to untreated patients. In general, for pharmaceutical compositions comprising one or more polypeptides, the amount of each polypeptide present in a dose ranges from about 100 μg to 5 mg per kg of host. Suitable volumes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### EXAMPLES

The following examples are presented to illustrate the present invention and to assist one of ordinary skill in making and using the same. The examples are not intended in any way to otherwise limit the scope of the invention.

### Example 1

### Elucidating the Roles and Interactions of Heat Shock Molecules Regulating Hsp70 Transcription and Expression

This example demonstrates the roles of molecules involved in regulating HSF binding to DNA through knock down and knock in studies. First, the proteins that make up the HSF complex were identified: HSF, Hsc4p and Hsc3p. The roles of these molecules were then examined using RNAi knock downs. The results of these studies show that, in non-shocked cells, Hsc4p represses DNA binding by HSF. Hsc3p appears to facilitate HSF DNA binding in stressed cells and to function as a co-activator for DNA binding in heat shocked cells. Knocking down Hsc4p allows HSF binding to promoters of the Hsp70 gene and activate transcription without heat shock.

I have established Drosophila cell lines (Schneider line 2) expressing HSF with a carboxyl terminal fusion of the IgG binding unit of protein A and the calmodulin-binding peptide (CBP) separated by a TEV-protease cleavage site (Forler et al. 2003). Purification of HSF complex (HSF-C) is achieved by first binding the extract (in the case of non-shocked cells a S10 cytosolic extract is prepared) to IgG Agarose for 2 hours and washing the resin, followed by elution of the complexes from the resin with TEV-protease. The isolated complexes are then bound to calmodulin Sepharose in the presence of calcium ions and eluted with EGTA. These TAP methods have been widely used in a large number of applications (Rigaut, et al. 1999; Puig, et al. 2001; Forler, et al. 2003).

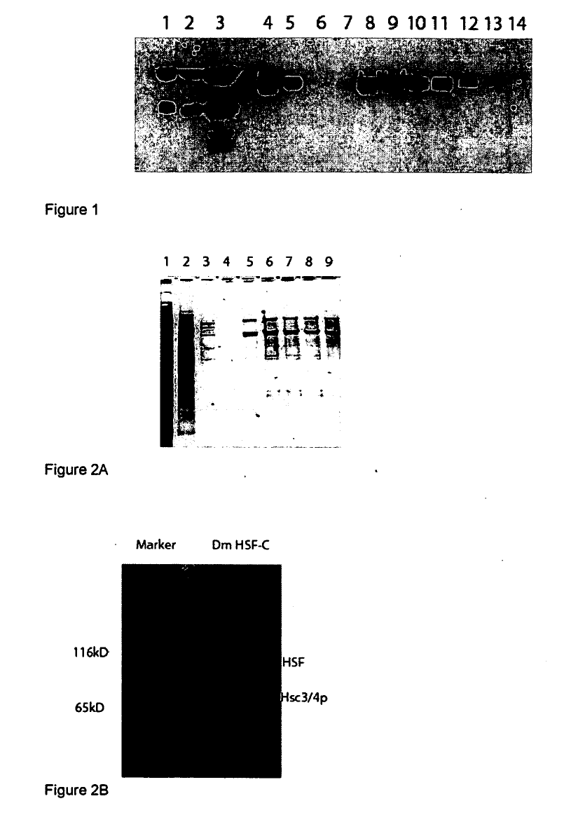

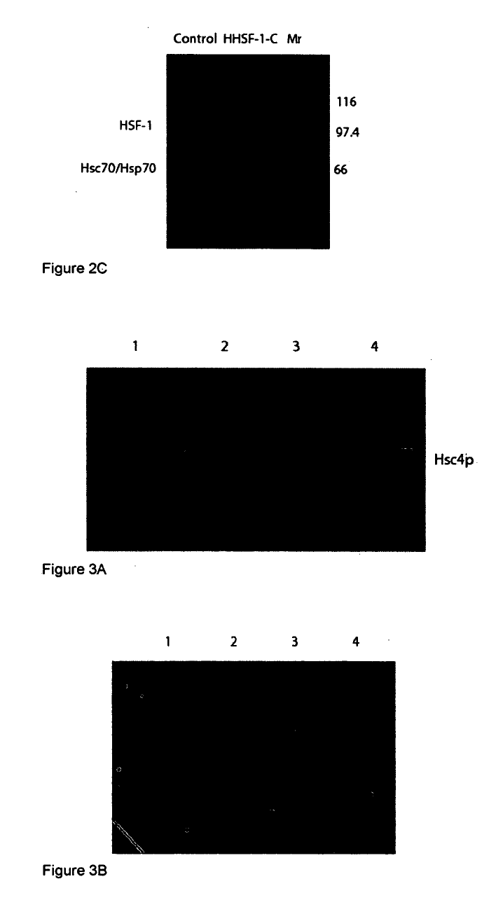

The success of this purification effort is shown in FIG. 1 (western blot probed with anti-HSF antibodies) and FIGS. 2a and b. In FIG. 1, the upper band of lanes 1-3 is the TAP-tagged HSF while the lower band is the endogenous HSF. After cleavage with TEV the upper tagged HSF is reduced in size by the loss of the IgG tag and the endogenous HSF is no longer present, lanes 4-14. An HSF complex (HSF-C) containing HSF and a doublet of two other proteins approximately 70 kD in size have been identified; FIG. 2a (silver stain) and FIG. 2b (coomassie stain). These three proteins were subjected to mass spectrometry (MS) analysis after purification from SDS-polyacrylamide gels. The higher molecular weight protein (110 kD) was identified as the heat shock transcription factor with a MOWSE score of 439 and 12 matching peptides. One of the 70 kD proteins was identified as Hsc4p with a MOWSE score of 464 (13 matching peptides) and 224, respectively, for two independent preparations. The other 70 kD protein was identified as Hsc3p with a MOWSE score of 135 (three matching peptides) and 78 for two independent preparations.

Other, lower molecular weight proteins were also found in somewhat lesser amounts to the Hsp4p and Hsc3p, FIG. 2a. FIG. 1 shows that the levels of TAP-tagged HSF is fundamentally the same as the endogenous HSF. Despite the fact that TAP-tagged HSF is under the control of the strong actin 5C promoter, it is not over-expressed in the stable cell line. This may be due to a strict limit on the level of HSF that is permitted to accumulate in the nucleus. If the levels of HSF were to become too high, it would cause inappropriate activation of DNA binding due to the high concentration of the factor. This is indeed what is observed when the nuclear localization sequence is deleted from the HSF resulting in cytological localization of the factor and a 20 to 100-fold increase in HSF levels compared to the nuclear factor concentration. Under these circumstances, the HSF binds DNA constitutively in the absence of heat stress.

Examination of the silver-stained Drosophila HSF-C gel (FIG. 2a) reveals a pattern of lower molecular weight proteins not easily seen in the coomassie stained gel. To determine the in vivo role of Hsc4p and Hsc3p in the heat-activation of HSF, RNAi methods have been used to knock down (KD) the expression of the cognates in S2 cells (Clemens, et al. 2000). Because the cognates have significant protein and nucleic acid homology within the coding region, traditional methods used in Drosophila to knock down mRNAs by targeting the coding region cannot be used. Examination of the 3′-untranslated regions revealed little similarities between the two cognates, so RNAi was prepared to specifically target these regions of the mRNAs.

Using Stressgen antibody SPA-822 that is supposed to react with Hsc 70 and Hsp70 the specificity of the RNAi knockdowns was examined by western blotting as shown in FIG. 3. RNAi directed against the 3′-UTR of Hsc4p greatly diminishes expression of Hsc 70 in non-shocked cells (FIG. 3 lane 1). When cells are heat shocked a small amount of protein running slower that Hsc70 is observed (FIG. 3, lane 2). This protein may correspond to the synthesis of Hsp70. RNA directed against the 3′UTR of Hsc3p did not significantly reduce the levels of the Hsc70 protein detected in the immunoblot. It is likely that Hsc4p is more abundant in the S2 cells than Hsc3p and therefore is not readily detected with this antibody. To show specificity for the knockdowns, Drosophila Hsc3p-specific antibodies were obtained from Babraham Bioscience (Cambridge, England, originally generated by Belinda Bullard clone MAC 143) (Elefant and Palter 1999). These antibodies should only detect Hsc3p in the knockdown experiments. Western analysis of RNAi knockdowns probed with this antibody is shown in FIG. 3b. The results show that RNAi directed against Hsc3p greatly reduced expression of Hsc3p but not Hsc4p. Similarly RNAi directed against Hsc4p did not reduce the levels of Hsc3p detected by the antibody.

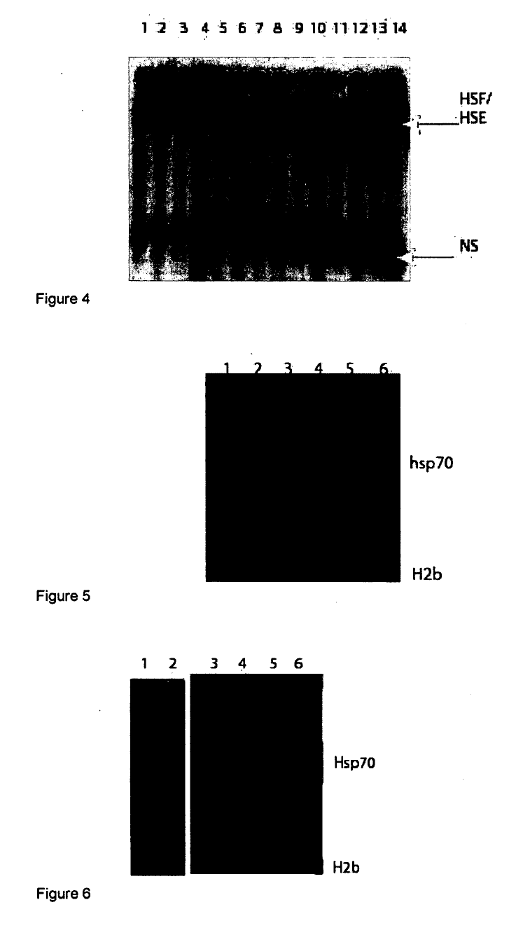

The effect of the KDs on DNA binding was first examined using the gel mobility shift assay. Briefly cells were treated with RNAi or transfected with the indicated DNA constructs and harvested four days later. Whole cell extracts were prepared and DNA binding carried out with a labeled heat shock element (HSE) double stranded oligonucleotide. The DNA binding results are shown in FIG. 4. Lanes 1 and 2 represent control cells, non-shocked and heat shocked, respectively. A small amount of DNA binding was apparent in the non-shocked extract with significant DNA binding with the heat-shocked extract. Lanes 3 and 4 contain extract derived from the Hsc3p KD cells. No DNA binding was observed in the non-shocked cells (lane 3) and a significant enhancement of DNA binding in the heat shocked extract (lane 4). Cells with the Hsc4p KD showed significant DNA binding in both the non-shocked case (lane 5) and when cells are heat shocked (lane 6). These results indicate that, in non-shocked cells, Hsc4p plays a repressive role in HSF DNA binding, while Hsc3p may attenuate DNA binding in stressed cells. The reduction in DNA binding of Hsc3p KD in non-stressed cells compared to control is probably not significant in that control cells normally do not show any DNA binding, but were slightly stressed in this experiment.

Clearly there is a significant difference in DNA binding activity between Hsc3p KD and Hsc4p KD. This result is very reproducible with the Hsc4p KD activating DNA binding of HSF in non-shocked cells. To explore the consequences of the HSF DNA binding activity in non-shocked cells with Hsc4p knocked down, the transcriptional activity of the Hsp70 genes was examined using primer-extension analysis with RNA isolated from the KD cells.

Primer extension analysis of Hsp 70 transcription in non-shocked and heat-shocked cells treated with RNAi is shown in FIG. 5. Control cells (lanes 1 and 2) show activation of Hsp70 transcription as a result of a ten-minute heat shock. The internal control is H2b RNA levels that are not altered significantly by heat shock (indicated as H2b in FIG. 5). Lanes 3 and 4 contain RNA from KD cells with RNAi against Hsc4p. Note the significant level of Hsp70 RNA, and this level is not significantly enhanced by heat-shock. This indicates that reduction in the levels of Hsc4p allows HSF to bind the promoters of Hsp70 genes and activate transcription without heat shock. Hsc3p KD does not result in the activation of Hsp70 gene transcription in non-shocked cells, but there is a small enhancement of Hsp70 transcription in heat-shocked cells. The transcription data agree with the DNA binding studies demonstrating that Hs4p represses the activity of HSF in non-shocked cells. When Hsc4p levels are reduced, HSF is able to bind DNA and activate transcription without heat shock.

FIG. 4 shows the effects of knocking in (KI) Hsc4p on the DNA binding and transcription activity of HSF. Lanes 7-12 compare non-shocked (odd numbered lanes) to heat-shocked (even numbered lanes). Cells transfected with wild-type Hsc4p (lanes 7 and 8) show levels of DNA binding similar to that of the controls with an enhancement in heat-shocked cells. Dominant negatively acting mutants K71S (lanes 9 and 10) and D206S (lanes 11 and 12) were transfected and non-shocked cells were compared to heat shocked cells at the level of HSF DNA binding (mutants provided by K. Palter, Temple University) (Elefant and Palter 1999). Heat shocked cells have elevated levels of DNA binding while non-shocked cells are comparable to controls. Thus these dominant negative mutations do not impair the repressive function on HSF DNA binding shown when wild-type Hsc4p it is knocked down in the cell. The dominant negative mutations occur in the ATP binding domain of Hsc4p and are defective in ATP-dependent clathrin-uncoating in vitro. Apparently this function of Hsc4p is not related to its role in repressing HSF in vivo.

The effects of the knock-ins on transcription of Hsp70 was also examined and the results are shown in FIG. 6. No activation of Hsp70 transcription was observed in non-shocked cells for either the wild-type protein or the two mutants (wild-type lanes 1 and 2; K71 S lanes 3 and 4; D206S lanes 5 and 6). Similarly, heat shock results in a normal induction of Hsp70 transcription for both the wild type and K71S mutant, however, the D206S mutant shows significant enhancement of Hsp70 transcription in heat shocked cells.

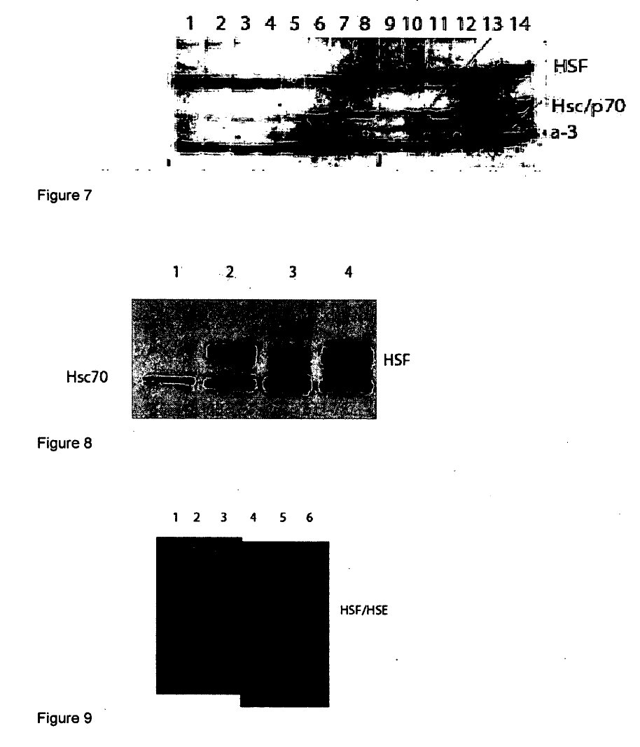

Examination of the levels of HSF itself in cells either KD'd with RNAi against Hsc3p or Hsc4p and KI's with wild type and the two mutant forms of Hsc4p was also performed and is shown in FIG. 7. The western blot shown in FIG. 7 was probed with a combination of three antibodies; ant-HSF, anti-Hsc70/Hsp70 (Stressgen SPA-822) and anti-karyohperin a3 (Fang, et al. 2001). HSF antibodies react with the upper band labeled HSF in the Figure. Hsc70/Hsp70 react with middle protein labeled Hsc70 in the Figure. Anti α-3 reacts with the fastest migrating protein in the Figure and serves as a loading control labeled a-3 in the Figure. Lanes 1 and 2 are from control cell either nonshocked (lane 1) or heat shocked (lane 2). Lanes 3 and 4 are from cells KD with Hsc3p RNAi, they both appear similar to the controls in the levels of the three proteins. Lanes 5 and 6 are from cells KD with Hsc4p RNAi. In this case, a dramatic reduction in HSF levels is observed in non-shocked cells when Hsc4p is knocked down (compare lanes 1 and 5). In heat shocked cells this effect is partially reversed with levels of HSF increasing but not at normal levels (compare lane 2 and 6).

This surprising result suggests a further role of Hsc4p in regulating HSF protein levels in addition to DNA binding. There is also some indication of Hsc70/Hsp70 increases in the Hsc4p KD in agreement with the transcription analysis supporting the finding of constitutive activation of HSF. Lanes 7 and 8 are derived form cells treated with RNAi against Drosophila CHIP a carboxyl terminus of Hsc70 interacting protein and an E3 ubiquitin ligase. This was done because Hsc4p is found in complex with HSF and the potential role that Hsc4p might play in the degradation of HSF. CHIP KD shows some increase in the levels of HSF but a more pronounced increase in Hsc70/Hsp70 in both non-shocked and heat shocked cells, indicating a role in the degradation of these proteins or a possible role in the activation of HSF. Preliminary analysis on the effects of CHIP KD in S2 cells demonstrates an enhancement of DNA binding in both non-shocked and heat shocked cells (FIG. 4. lanes 13 and 14).

Knocking in Hsc4p and the two dominant negative mutants has little effect on HSF levels in the cell (FIG. 7, lanes 9-14). There is a significant increase in the levels of Hsc70/Hsp70 in cells expressing both mutant Hsc4p genes (FIG. 7, lanes 11-14). The experiments shown in FIG. 7 do not distinguish between Hsp70 or Hsc70 proteins, so it is not known if the induced protein is Hsp70. Analysis of the Hsp70 mRNAs levels by primer extension did not demonstrate any significant activation of Hsp70 gene transcription in non-shocked cells FIG. 6 (lanes 3-6). There is an increase in Hsp70 synthesis when D206S is expressed in heat-shocked cells (FIG. 6, lanes 5 and 6). Earlier experiments by Elefant and Palter showed that in larvae expressing the dominant negative Hsc4p cognates, Hsp70 proteins were induced in the absence of heat shock. The antibodies used were specific for Hsp70 and not reactive with Hsc70 (rat mAb 7.FB, Lindquist). Unfortunately these antibodies are no longer available, so it is not possible to compare results directly.

Two other aspects of HSF-C have been examined, at least preliminarily. Does the complex stay together or change during heat shock and does the purified HSF-C have activity in vitro? Two experiments have been carried out. An s2 cell line expressing Hsc4p with a FLAG tag on the carboxyl terminus was established. Whole cell extracts prepared from non-shocked and heat shocked cells and immunoprecipitated with anti-HSF antibodies. The results of western blotting are shown in FIG. 8. The blot was probed with antibodies reactive against both HSF and FLAG. Lanes 1 and 3 contain the supernatant of the immunoprecipitation; lanes 2 and 4 are the immunoprecipitated samples. Non-shocked cells show that Hsc4p is in a complex with HSF as expected. Analysis of heat-shocked cells also demonstrates that Hsc4p is retained during heat shock in the HSF complex. Lanes 1 and 3 (immunoprecipitation supernatant) demonstrate that the anti-HSF antibodies are effective in removing most of the detectable HSF from the extract.

The activities of the complex, whether the complex can sense temperature and bind DNA in vitro, or are other components needed, was explored. Purified HSF-C from non-shocked cells and partially purified Hsc4p from the FLAG tagged cell line were prepared. Purification of the FLAG tagged Hsc4p was carried out using Ni-Agarose taking advantage of the histidine tag next to the FLAG tag on the carboxyl terminus of the protein. Shown in FIG. 9 are preliminary results obtained from an experiment aimed at determining whether the complex can respond to temperature in vitro and activate DNA binding on its own or with the help of the Hsc4p cognate. Proteins are assembled along with DNA binding components (labeled HSF, dldC and BSA) on ice, then incubated for three minutes at either 25° C. (non-shocked) or at 36° C. (heat shocked). The samples are returned to ice for twenty minutes to allow the “activated” complex to bind DNA. EMSA was used to determine the level of DNA binding.

In FIG. 9, lanes 1-3 are non-shocked, lanes 4-6 are heat shocked. Lanes 1 and 4 contain the purified HSF-C, lanes 2 and 5 contain the purified Hsc4p, lanes 3 and 6 contain a combination of both preparations. The addition of Hsc4p clearly stimulated the DNA binding of HSF-C at both 25° C. and 36° C. However, the DNA binding activity at 36° C. is nearly 20-fold higher than at 25° C. Although the results are preliminary, they indicate that it may be possible to reconstruct HSF temperature sensing in vitro from purified components.

### REFERENCES

### Cited in Background and in Example 1

Ahn, S. G., et al. (2005). Biochem J 392(Pt 1): 145-52.
Clemens, J. C., et al. (2000). PNAS 97(12): 6499-6503.
Elefant, F. and K. ‘R. Palter (1999). Mol Biol Cell 10(7): 2101-17.
Fang, X. D., et al. (2001). Development 128(17): 3349-3358.
Farkas, T., et al. (1998). Mol Cell Biol 18(2): 906-18.
Forler, D., et al. (2003). Nat Biotechnol 21(1): 89-92.
Goodson, M. L. and K. D. Sarge (1995). J Biol Chem 270(6): 2447-50.
Lindquist, S, and E. A. Craig (1988). Annu Rev Genet. 22: 631-77.
Puig, O., et al. (2001). Methods 24(3): 218-29.
Rabindran, S. K., et al. (1991). Proc Natl Acad Sci USA 88(16): 690-610.
Rabindran, S. K., et al. (1993). Science 259(5092): 230-4.
Rabindran, S. K., et al. (1994). Mol Cell Biol 14(10): 6552-60.
Rigaut, G., et al. (1999). Nat Biolechnol 17(10): 1030-2.
Sarge, K. D., et al. (1993). Mol Cell Biol 13(3): 1392-407.
Zhong, M., et al. (1998). Mol Cell 2(1): 101-8.
Zuo, J., et al. (1994). Mol Cell Biol 14(11): 7557-68.

### Example 2

### Mechanism of Regulation of HSF DNA Binding

This example demonstrates that temperature sensing by HSF is a function of the association of the HSF with two Hsc proteins: Hsc3p, which functions as a co-activator, and Hsc4p, which functions as a repressor in non-shocked cells.

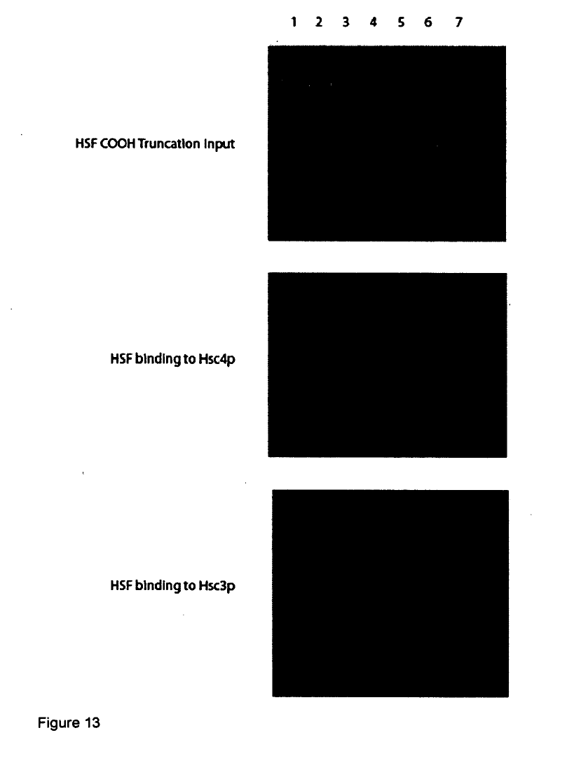

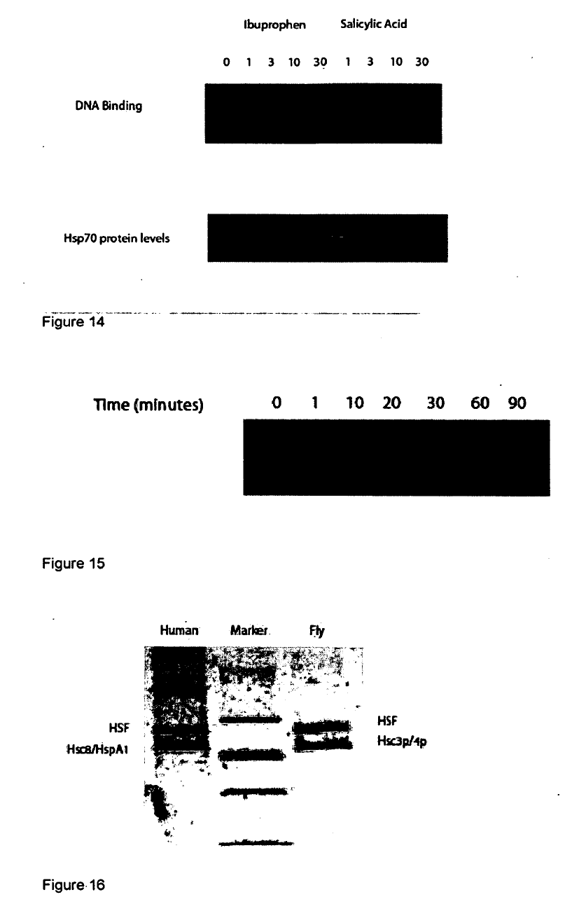

The domains of HSF that interact with Hsc3p and Hsc4p have been determined and found to be primarily located to the oligomerization domain of HSF. Shown in FIG. 13 is a protein-protein interaction analysis. HSF was synthesized using a wheat germ coupled transcription/translation synthesis system with S35 methionine. The templates used were generated with PCR and primers that allowed specific truncation of the carboxyl terminus of the HSF resulting in progressively smaller labeled HSF molecules, shown as input. Bacculovirus expressed Hsc3p and Hsc4p were combined with the labeled HSF molecules and incubated overnight at 4° C. Talon chelating beads were added and the reactions continued for 90 minutes on a rotating mixer. These beads will bind the Hsc3p/4p proteins but not the labeled HSF. The beads were washed with buffer containing 0.35M KCl three times for ten minutes. The bound proteins were then eluted with LSB and analyzed on a 4-20% gradient gel. The gels were fixed and impregnated with Autofluor and dried prior to exposure to film.

It is clear that HSF is bound to the cognates up to residue 145. Between residues 245 to 145 is the oligomerization domain of the HSF. This domain is required for HSF to adopt the higher order trimer structure needed for high affinity DNA binding. Truncation at residue 145 removes the oligomerization domain but retains the DNA binding domain. Hsc4p shows ally no binding to the DNA binding domain while Hsc3p maintains a very low level of interaction.

These results are significant, for they suggest the mechanism of Hsc4p repressing DNA binding could be by blocking oligomerization of the factor in non-stressed cells. When Hsc4p is removed from cells by RNAi methods, the HSF adopts the higher order trimer structure and binds DNA. Hsc3p functions as a cofactor aiding in DNA binding when cells are heat shocked, suggesting that, at elevated temperature, the conformation of the HSF-C is dynamically altered with the help of Hsc3p.

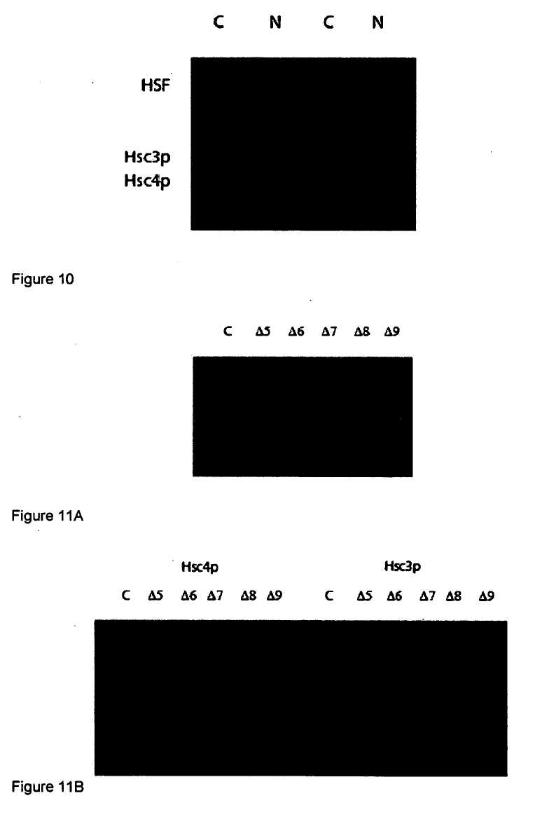

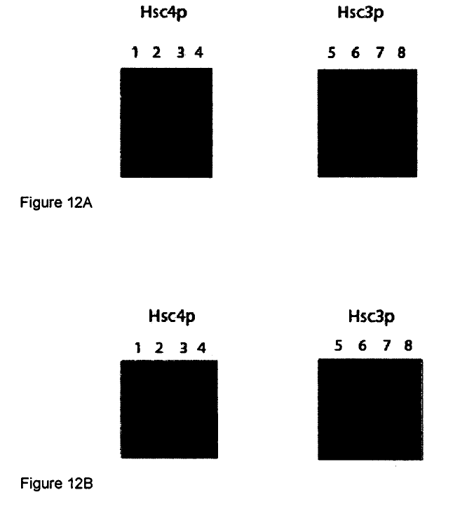

The above observations suggest that Hsc4p could simply dissociate upon heat shock, allowing the HSF to bind with the aid of Hsc3p. To assess this possibility, cells stably expressing HSF with a streptavidin-binding domain were used to compare the complexes from normally growing cells to heat shocked cells. Cells non-shocked and heat shocked were fractionated into nuclear and cytosolic extracts and HSF-C pulled out with streptavidin Agarose beads. Proteins were subjected to SDS-PAGE and western blotting. The westerns were probed with a combination of antibodies reactive against HSF, Hsc3p and Hsc4p. Shown in FIG. 10 are the results of this analysis.

The Drosophila HSF is primarily nuclear in both non-shocked and heat shocked cells. Biochemical fractionation methods, however, lead to the leakage of HSF into the cytosol when cells are lysed. In all cases, the cytosolic form of the factor cannot bind DNA. Note the presence of both Hsc3p and Hsc4p in the cytosolic non-shocked fraction and the nuclear heat shocked fraction indicating the complex does not dissociate upon heat shock. However also note that the cytosolic non-binding complex in heat shocked cells lacks Hsc3p that is needed in vivo to aid in the activation of DNA binding.

These studies show that temperature sensing by HSF is a function of the association of the HSF with two Hsc proteins: Hsc3p. which functions as a co-activator, and Hsc4p, which functions as a repressor in non-shocked cells. Both cofactors remain associated with activated HSF through interactions with the oligomerization domain of the HSF. It is apparent that temperature modifies the structure of the Hsc-HSF complex in such a way that the HSF can trimerize and bind DNA.

### Example 3

### Activation of HSF to Produce Neuroprotective Levels of Hsp70

This example demonstrates the ability to activate the HSF without cellular stress via small molecules. The goal here is to activate HSF to produce adequate levels of Hsp70 that can provide neuroprotective functions to the human brain. To begin these studies, two common non-steroidal anti-inflammatory drugs (NSAIDs), ibuprofen and salicylic acid, were tested. These molecules have been previously shown to stimulate HSF DNA binding in mammalian and Drosophila cells. The findings presented here differ from those previously published. As reported here, both salicylic acid and ibuprofen can stimulate HSF DNA binding, Hsp70 transcription and Hsp70 protein synthesis. FIG. 13 shows the concentration of salicylic acid and ibuprofen that are optimal for induction of DNA binding and Hsp70 protein synthesis in Drosophila cultured cells. It is clear that both DNA binding and Hsp70 protein synthesis have identical optimums.

Ibuprofen shows a narrow optimum between 3-10 mM, whereas salicylic acid is quite broad and can function at low concentrations, such as 1 mM. In addition to DNA binding and protein synthesis, Hsp70 transcription is induced by salicylic acid for a significant period of time. Shown in FIG. 15 is a time course of Hsp70 transcription in response to 3 mM salicylic acid.

Thus, low levels of NSAIDs can be used to stimulate neuroprotective amounts of Hsp70 transcription and protein synthesis.

### Example 4

### Screening for Additional Agents that Elicit Neuroprotective Levels of Hsp70

This example demonstrates the methods that can be used to identify further agents, such as small molecules or other NSAIDs, that can increase Hsp70 synthesis in non-stressed cells. A rapid, high-throughput assay uses a reporter gene operably linked to the hsp70 gene to create a reporter plasmid that responds to activation of Hsp70 synthesis by producing a detectable signal, such as fluorescence, within 10-20 minutes. Candidate agents are brought into contact with cultured cells containing the reporter construct. The cells can be cultured under standard conditions, without stress conditions. For example, the cells are typically cultured at 37° C.

The pGL4.26[luc2/minP/Hygro]), pGL4.27 [luc2P/minP/Hygro] and pGL4.28[luc2CP/minP/Hygro] vectors can be used for cloning the Hsp70 regulatory element to monitor/screen against the compounds library. The main difference of these 3 vectors is the half-life of the luciferase reporter protein due to absence or presence of protein degradation signal(s). Promega (Madison, Wis.) has various other pGL4 vectors with different selectable markers (neo, puro, hygro), reporters (firefly, Renilla), etc., which can also be used.

To minimize variation during screening, it is preferable to establish a stable cell line carrying the screening reporter (instead of using transient transfection). In addition, a reference reporter should be considered for normalization purposes (in a dual-luciferase assay format). The Renilla luciferase gene is used commonly for this purpose. One of the pGL4.79-81-[hRluc/Neo] and pGL4.82-84[hRluc/Puro] vectors could be used. A reference regulatory element (e.g. TK promoter or SV40 promoter or others) would first be cloned in front of the Renilla luciferase reporter.

### Example 5

### Further Data Relating to Purification of HSF Complex and Protein Interactions

This example provides further data relating to the studies described in Example 1 above. FIG. 16 is an SDS gel showing purification of HSF complex from human and fly cells. Human HK293 cells were transfected with human HSF tagged with a streptavidin binding protein tag and the calmodulin binding domains. Purification of human HSF-C was achieved by tandem affinity chromatography with streptavidin resin and calmodulin resin. The Drosophila HSF-C (fly) was purified by tagging the HSF with the IgG binding domain of S. aureus protein A and the calmodulin binding domain separated by a TEV protease cleavage site. A stable cell line was generated expressing the tagged HSF. The fly HSF was purified by tandem affinity chromatography using IgG Sepharose, TEV cleavage and calmodulin affinity chromatography. Samples were subjected to SDSpolyacrylamide gel electrophoresis and the gel stained with silver. The identities of the labeled proteins were determined by Mass spectrometry. Both human and fly HSF-C contain the hsc70 cognate (Hsc8 for the human and Hsc4p for the fly).

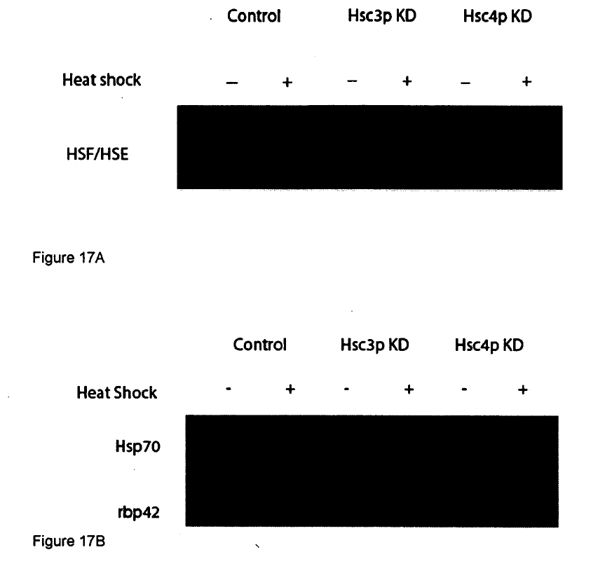

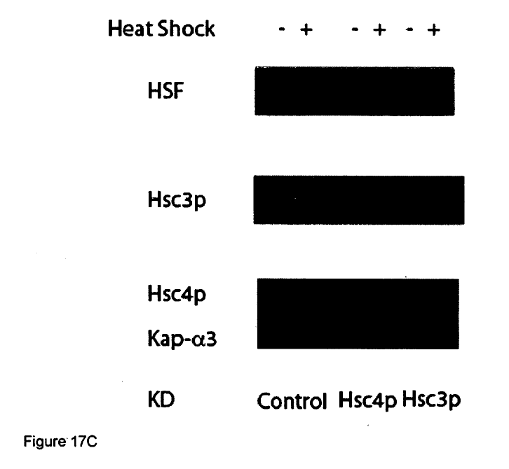

FIGS. 17A-17C show the effects of knocking down Hsp3p (Hsc72/Bip) and Hsc4p (Hsc70) on the heat shock response in Drosophila in vivo. DNA binding activity of HSF was measured by electrophoretic mobility-shift assay (EMSA). A labeled DNA corresponding to the recognition site of the HSF (heat shock element HSE) was mixed with nuclear extracts prepared from normal cells (Control), either no-shocked or heat shocked, and the reaction was subjected to native gel electrophoresis. The results are shown in FIG. 17A. Robust DNA binding is observed when the control cells are heat shocked. Reduced DNA binding is observed in heat shock cells where Hsc3p is knocked down and significant DNA in both non-shocked and heat-shocked cells occurs when Hsc4p is knocked down.

Hsp70 transcription was monitored by primer extension analysis. As a control, included in the reactions was the ribosomal binding protein 42 (rbp42). Robust heat shock dependent transcription was exhibited in the control cells, with a reduction in Hsp70 transcription in Hsc3p knocked down cells and hsp70 transcription in both non shocked and heat shocked cells when hsc4p was knocked down. These results (FIG. 17B) support the idea that hsc3p is a co-activator of HSF activity in heat shocked cells and hsc4p is a repressor of HSF activity in non shocked cells.

HSF levels are dramatically reduced when Hsc4p is knocked down, suggesting that the active form of HSF is degraded rapidly in non-stressed cells. Perhaps this functions as a mechanism to limit the concentration of active HSF when cells are not stressed, when control cells are heat shocked the HSF is stabilized. The series of western blots shown in FIG. 17C demonstrates the specificity of the individual knock downs and presents a control of protein levels with Kap-a3 nuclear transporter molecule.

### Example 6

### HSF Binding Domains on Hsc4p and Hsc3p

This example provides a more defined view of the interactions between the two cognates, Hsc4p and Hsc3p, and the oligomerization domain of the HSF. FIG. 11A shows the S35 HSF molecules used in the binding reactions. C represents the N-245 amino acid segment of HSF containing the DNA binding domain (residues N-145) and the oligomerization domain (residues 146-245) with no internal deletions. The D series represents a series of contiguous internal deletions of 30 amino acids constructed through out the entire HSF molecule. The oligomerization domain begins within D5 and continues through D9. Specifically the residues deleted for each deletion are as follows: D5; 121-150, D6; 151-180, D7; 181-210, D8; 211-240 and D9; 241-270.

The binding reactions are performed using the indicated in vitro synthesized HSF deletion and control and bacculovirus produced Hsc3p and Hsc4p. The proteins are combined overnight at 4° C., Talon metal binding beads added and the sample further incubated at 4° C. for 90 minutes with agitation. The beads are washed and eluted with Laemmli sample buffer and subjected to SDS-polyacrylamide gel electrophoresis. The gels are then impregnated with a fluor solution, dried and exposed to film.

The results (FIG. 11B) show that an important interaction occurs between Hsc4p and D7 (residues 181-210) and Hsc3p a broader interaction with D7 and D8 (residues 211-240) although the D8 interaction is less strong than D7.

### Example 7

### HSF Binding Domains on Hsc4p and Hsc3p

This example demonstrates that HSF binds to the amino terminal portion of both cognates (Hsc4p and Hsc3p). Carboxyl terminal deletions of Hsc4/3p were synthesized in vitro and labeled with S35. These proteins were mixed with full length HSF produced by bacculovirus expression in High-five cells. The binding reactions were continued overnight at 4° C. followed by the addition of Talon metal binding beads for 90 minutes with agitation. Samples were washed and eluted with Laemmli sample buffer and subjected to SDS-polyacrylamide gel electrophoresis. The gels were impregnated with fluor, dried and exposed to film. Input to the binding reactions is shown in FIG. 12A. The results of protein binding are shown in FIG. 12B. Lanes 14 contain Hsc4p and lanes 5-8 contain Hsc3p. Lanes 1 and 5 contain full length proteins, lanes 2 and 6 contain carboxyl terminal truncations at residues 506 and 501 respectively. Lanes 3 and 7 contain carboxyl terminal truncations at residues 406 and 410, respectively. Lanes 4 and 8 contain carboxyl terminal truncations at residues 306 and 311, respectively. Clearly HSF binds to the amino terminal portion of both cognates, specifically the first 300 amino acids, that represents the ATPase domains of the proteins.

From the foregoing it will be appreciated that, although specific embodiments of the invention have been described herein for purposes of illustration, various modifications may be made without deviating from the spirit and scope of the invention. Accordingly, the invention is not limited except as by the appended claims.

Throughout this application various publications are referenced. The disclosures of these publications in their entireties are hereby incorporated by reference into this application in order to describe more fully the state of the art to which this invention pertains.

What is claimed is:

1. A method of increasing Hsp70 synthesis in a cell in the absence of stress, the method comprising contacting the cell with an effective amount of an agent that disrupts the interaction of Hsc70 with heat shock transcription factor (HSF).

2. A method of activating a heat shock response in a cell in the absence of stress, the method comprising contacting the cell with an effective amount of an agent that disrupts the interaction of Hsc70 with HSF.

3. The method of claim 2, wherein the agent comprises a non-steroidal anti-inflammatory drug (NSAID).

4. The method of claim 3, wherein the NSAID comprises salicylic acid or ibuprofen.

5. The method of claim 2, wherein the agent disrupts binding between Hsc70 and the oligomerization domain of HSF.

6. The method of claim 2, wherein the agent comprises an RNAi directed at Hsc70.

7. The method of claim 2, wherein the heat shock response comprises increased transcription of Hsp70.

8. The method of claim 2, wherein the heat shock response comprises increased synthesis of Hsp70.

9. The method of claim 2, wherein the cell is in vivo.

10. The method of claim 2, wherein the cell is in vitro.

11. The method of claim 2, wherein the contacting occurs while the cell is maintained at a temperature of 28 to 39° C.

12. The method of claim 11, wherein the temperature is about 37° C.

13. The method of claim 3, wherein the effective amount of NSAID is between 0.01 and about 10 mM.

14. The method of claim 13, wherein the effective amount of NSAID is about 0.5 to about 3 mM.

15. The method of claim 2, wherein the contacting occurs for about 1 to about 90 minutes.

16. The method of claim 15, wherein the contacting occurs for about 60 minutes.

17. A method of protecting cells from neurodegenerative proteins in the brain of a mammalian subject, the method comprising:
administering to the subject an effective amount of a non-steroidal ant-inflammatory drug (NSAID),
wherein the amount of NSAID is sufficient to increase binding of heat shock transcription factor (HSF) to DNA, increase transcription of Hsp70, or to increase synthesis of Hsp70 in brain cells of the subject.

18. The method of claim 17, wherein the administering is oral.

19. A method of identifying an agent that protects neural cells from neurodegenerative proteins, the method comprising:
(a) contacting cultured cells with a candidate agent; and
(b) assaying the cultured cells for levels of Hsp70 protein,
wherein an increase in levels of Hsp70 protein is indicative of a neuroprotective agent.

20. The method of claim 19, wherein the cells are mammalian cells or Drosophila cells.

21. The method of claim 19, wherein the cultured cells contain a reporter plasmid that responds to activation of Hsp70 synthesis by producing a detectable signal and the assaying comprises measuring levels of the detectable signal.
